(19) United States
(12) Patent Application Publication
(10) Pub. No.: US 2018/0104473 A1
(43) Pub. Date: Apr. 19, 2018

(54) COCHLEAR ELECTRODE ARRAY WITH A FLEXURAL INFLECTION POINT

(71) Applicant: ADVANCED BIONICS AG, Staefa (CH)

(72) Inventor: Kate Purnell, Valencia, CA (US)

(21) Appl. No.: 15/567,506

(22) PCT Filed: May 5, 2015
(86) PCT No.: PCT/US2015/029281

§ 371 Date: Oct. 18, 2017

(51) Int. Cl. A61N 1/05; A61N 1/36

(52) U.S. Cl. CPC A61N 1/0541; A61N 1/36038

(57) ABSTRACT

A cochlear implant device includes a flexible electrode array for insertion into a cochlea. The flexible electrode array includes a number of electrodes; a number of electrical wires coupled to the electrodes; and a flexural inflection point positioned on the flexible electrode array such that, when implanted, is situated at a basal turn of the cochlea.

### BACKGROUND

In human hearing, hair cells in the cochlea respond to sound waves and produce corresponding auditory nerve impulses. These nerve impulses are then conducted to the brain and perceived as sound.

Damage to the hair cells results in loss of hearing as sound waves received by the cochlea are not transduced into auditory nerve impulses. This type of hearing loss is called sensorineural deafness. To overcome sensorineural deafness, cochlear implant systems have been developed, which include an electrode array implanted in the cochlea. These cochlear implant systems bypass the defective or missing hair cells by directly stimulating the auditory nerve via the implanted electrode array. This stimulation generates auditory nerve impulses, which are transmitted from the auditory nerve to the brain. This leads to the perception of sound and provides at least partial restoration of hearing function.

### BRIEF DESCRIPTION OF THE DRAWINGS

The accompanying drawings illustrate various examples of the principles described herein and are a part of the specification. The illustrated examples are merely examples and do not limit the scope of the claims.

FIG. 1 is a diagram showing a cochlear implant system, according to one example of principles described herein.

FIG. 2 is a diagram showing external components of a cochlear implant system, according to one example of principles described herein.

FIG. 3 is a diagram showing the internal components of a cochlear implant system, according to one example of principles described herein.

FIG. 4 is a diagram of an example of an implantable device during an insertion procedure according to one example of principles described herein.

FIG. 5 is a graph depicting the insertion forces of an implantable device according to one example of principles described herein.

FIG. 6 is a diagram of an electrode array of an implantable device with a flexural inflection point according to one example of principles described herein.

FIG. 7 is a diagram of an electrode array of an implantable device with a number of flexural inflection points according to another example of principles described herein.

FIG. 8 is a diagram of an electrode array of an implantable device with a number of flexural inflection points according to another example of principles described herein.

FIG. 9 is a flow diagram of a method for implanting a lead of an implantable device with a flexural inflection point according to one example of principles described herein.

Throughout the drawings, identical reference numbers designate similar, but not necessarily identical, elements.

### DETAILED DESCRIPTION

As mentioned above, individuals with hearing loss can be assisted by a number of hearing devices, including cochlear implants. Typical cochlear implant systems are made up of both external and implanted components. The external components detect environmental sounds and convert the sounds into acoustic signals. These acoustic signals are separated into a number of parallel channels of information, each representing a band of frequencies within the perceived audio spectrum. Each channel of information is conveyed to a subset of auditory nerve cells that transmit information about that frequency band to the brain. Those nerve cells are arranged in an orderly tonotopic sequence, from the highest frequencies at the proximal end of the cochlear spiral to progressively lower frequencies towards the apex. A flexible electrode array is inserted into the cochlea and has a number of electrodes that correspond to the tonotopic organization of the nerve cells in the cochlea.

When the cochlear implant is placed in a patient's cochlea, it is important that the implant be inserted to a proper depth such that the stimulating electrodes on the implant are proximal to corresponding nerve cells in the tonotopic sequence. If the implant is not inserted to a sufficient depth, electrodes intended to stimulate specific corresponding never cells in the tonotopic sequence may, instead, stimulate different cells corresponding to a different frequency band than intended. As a result, any auditory information conveyed will seem to have the wrong frequency as compared to how the represented noise should sound. Alternatively, if pressure is applied to the implant after the implant has reached the proper depth, this pressure may cause trauma to the cochlea.

Similarly, once the implant is properly placed in the patient's cochlea, the implant should retain its position. If the implant moves or extrudes, similar issues may result in correctly conveying sound frequencies to the patient. Additionally, movement or extrusion of the implant may cause trauma to the cochlea or even a need to reposition the implant in a subsequent procedure.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “an example,” or similar language means that a particular feature, structure, or characteristic described in connection with the example is included in at least that one example, but not necessarily in other examples. The various instances of the phrase “in one example” or similar phrases in various places in the specification are not necessarily all referring to the same example.

As used in the present specification and in the appended claims, the term “a number of” or similar language may include any number including one to infinity; zero not being a number, but the absence of a number.

A flexible electrode array may be a thin, elongated body with a proximal end and a distal end. A number of electrodes, for example numbering between 6 and 30, are disposed on the distal end of the body. The electrodes may be longitudinally disposed and separately connected stimulating electrode contacts. The number of electrodes constitutes an electrode array. According to one illustrative example, the flexible electrode array may be constructed of a biocompatible silicone, platinum-iridium wires, and platinum electrode contacts such that the distal end of the flexible electrode array has the flexibility to curve around the helical interior of the cochlea. In use, the flexible electrode array may have a tendency to straighten, but by adding a number of flexural inflection points, the tendency of the flexible electrode array to straighten may be reduced.

To place the flexible electrode array into the cochlea, the flexible electrode array may be inserted through a cochleostomy or via a surgical opening made in the round window of the cochlea. The flexible electrode array is inserted through the opening into the scala tympani, one of the three parallel ducts that make up the spiral-shaped cochlea. For example, the flexible electrode array may be inserted into the scala tympani duct in the cochlea to a depth of about 13 to 30 millimeters (mm).

When in use, the electrode array delivers electrical current into the fluids and tissues immediately surrounding the individual electrode contacts to create transient potential gradients which, if sufficiently strong, cause the nearby auditory nerve fibers to generate action potentials. The auditory nerve fibers branch from cell bodies located in the spiral ganglion, which ganglion lies in the modiolus, adjacent to the inside wall of the scala tympani. The density of electrical current flowing through the tissues and fluids may be highest near the electrode contact that is the source of such current. Consequently, stimulation at one electrode contact site tends to selectively activate those spiral ganglion cells and their auditory nerve fibers that are closest to that contact site.

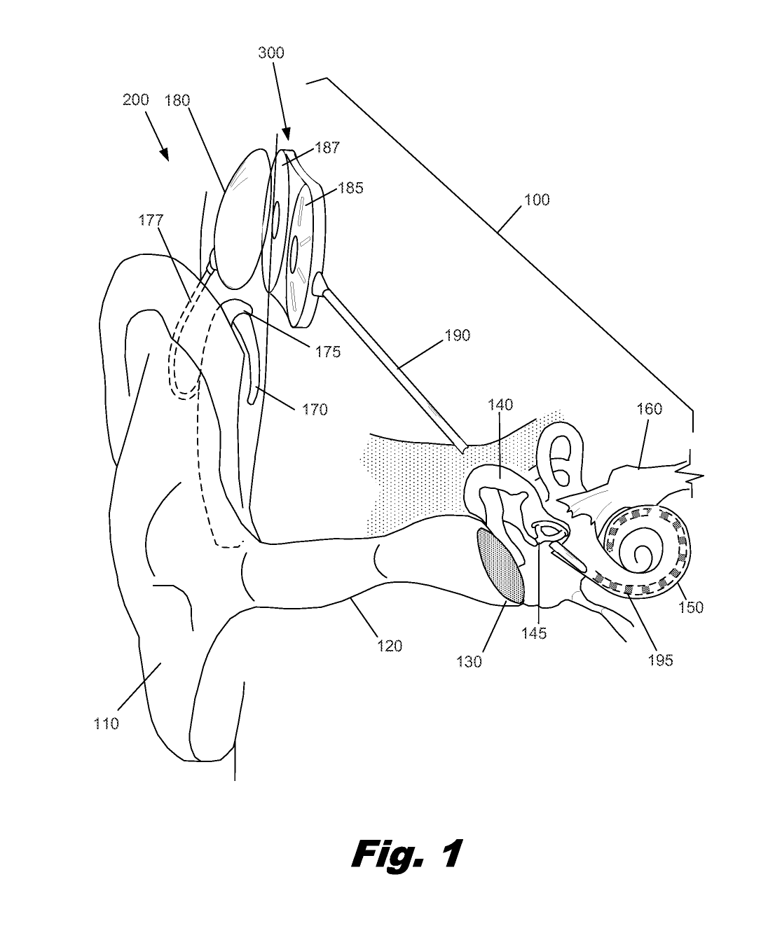

FIG. 1 is a diagram showing one illustrative example of a cochlear implant system (100) having a cochlear implant (300) with a flexible electrode array (195) that is surgically placed within the patient's auditory system.

In normal hearing, sound enters the external ear, or pinna, (110) and is directed into the auditory canal (120), where the sound wave vibrates the tympanic membrane (130). The motion of the tympanic membrane is amplified and transmitted through the ossicular chain (140), which consists of three bones in the middle ear. The third bone of the ossicular chain (140), the stirrup (145), contacts the outer surface of the cochlea (150) and causes movement of the fluid within the cochlea. Cochlear hair cells respond to the fluid-borne vibration in the cochlea (150), and trigger neural electrical signals that are conducted from the cochlea to the auditory cortex by the auditory nerve (160).

In many cases, deafness is caused by the absence or destruction of the hair cells in the cochlea, i.e., sensorineural hearing loss. In the absence of properly functioning hair cells, there is no way auditory nerve impulses can be directly generated from ambient sound. Thus, conventional hearing aids, which amplify external sound waves, provide no benefit to persons suffering from complete sensorineural hearing loss.

By contrast, a cochlear implant (300) does not simply amplify sound, but works by directly stimulating the auditory nerve (160) with electrical impulses delivered by electrodes implanted in the cochlea. Because this direct electrical stimulation bypasses the defective cochlear hair cells that normally transduce acoustic energy into electrical energy, a cochlear implant can provide a sense of sound to a person who is profoundly deaf or severely hard of hearing, despite the absence of functioning hair cells.

External components (200) of the cochlear implant system can include a Behind-The-Ear (BTE) unit (175), which contains the sound processor and has a microphone (170), a cable (177), and a transmitter (180). The microphone (170) picks up sound from the environment and converts it into electrical impulses. The sound processor within the BTE unit (175) selectively filters and manipulates the electrical impulses and sends the processed electrical signals through the cable (177) to the transmitter (180). The transmitter (180) receives the processed electrical signals from the processor and transmits them to the implanted antenna (187) by electromagnetic transmission. In some cochlear implant systems, the transmitter (180) is held in place by magnetic interaction with a magnet in the center of the underlying antenna (187).

The components of the cochlear implant (300) include an internal processor (185), an antenna (187), and a cochlear lead (190) which terminates in an electrode array (195). The internal processor (185) and antenna (187) are secured beneath the user's skin, typically above and behind the pinna (110). The antenna (187) receives signals and power from the transmitter (180). The internal processor (185) receives these signals and performs one or more operations on the signals to generate modified signals. These modified signals are then sent along a number of delicate wires which pass through the cochlear lead (190). These wires are individually connected to the electrodes in the electrode array (195). The electrode array (195) is implanted within the cochlea (150), and provides electrical stimulation to the auditory nerve (160).

The cochlear implant (300) stimulates different portions of the cochlea (150) according to the frequencies detected by the microphone (170), just as a normally functioning ear would experience stimulation at different portions of the cochlea depending on the frequency of sound vibrating the liquid within the cochlea (150). This allows the brain to interpret the frequency of the sound as if the hair cells of the basilar membrane were functioning properly.

FIG. 2 is an illustrative diagram showing a more detailed view of the external components (200) of one example of a cochlear implant system. External components (200) of the cochlear implant system include a BTE unit (175), which comprises a microphone (170), an ear hook (210), a sound processor (220), and a battery (230), which may be rechargeable. The microphone (170) picks up sound from the environment and converts it into electrical impulses. As discussed above, the sound processor (220) selectively filters and manipulates the electrical impulses and sends the processed electrical signals through a cable (177) to the transmitter (180). A number of controls (240, 245) adjust the operation of the processor (220). These controls may include a volume switch (240) and program selection switch (245). The transmitter (180) receives the processed electrical signals from the processor (220) and transmits these electrical signals and power from the battery (230) to the cochlear implant by electromagnetic transmission.

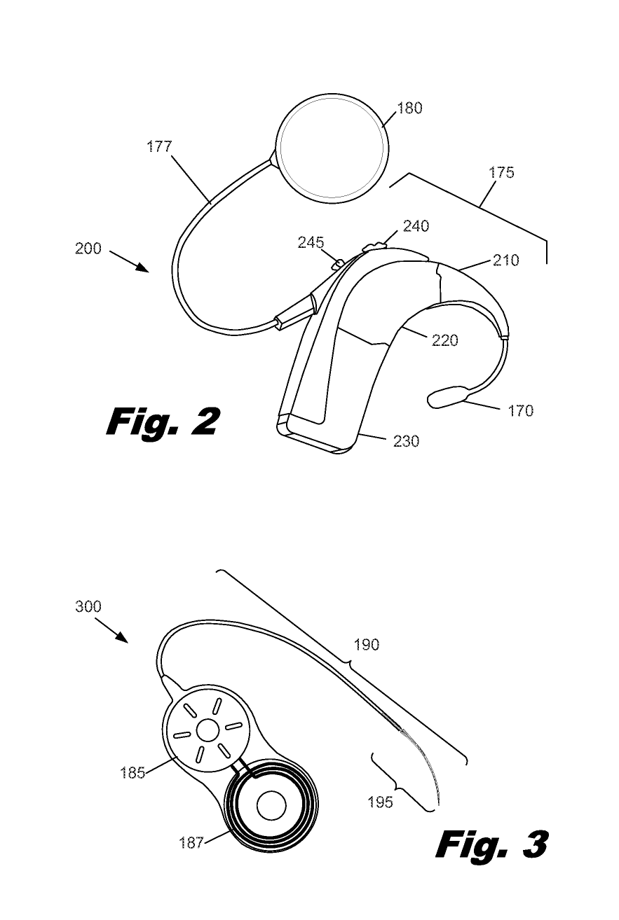

FIG. 3 is an illustrative diagram showing one example of a cochlear implant (300), including an internal processor (185), an antenna (187), and a cochlear lead (190) having an electrode array (195). The cochlear implant (300) is surgically implanted such that the electrode array (195) is internal to the cochlea, as shown in FIG. 1. The internal processor (185) and antenna (187) are secured beneath the user's skin, typically above and behind the pinna (FIG. 1, 110), with the cochlear lead (190) connecting the internal processor (185) to the electrode array (195) within the cochlea. As discussed above, the antenna (187) receives signals from the transmitter (180) and sends the signals to the internal processor (185). The internal processor (185) modifies the signals and passes them along the appropriate wires to activate one or more of the electrodes within the electrode array (195). This provides the user with sensory input that is a representation of external sound waves sensed by the microphone (170).

As noted above, the cochlear implant should be inserted to a proper depth, and not beyond, so as to correctly convey signals representing detected sound in each frequency band to corresponding nerve cells along the tonotopic sequence of the cochlea. Similarly, once in place, the cochlear implant should retain that positioning without movement or extrusion relative to the cochlea.

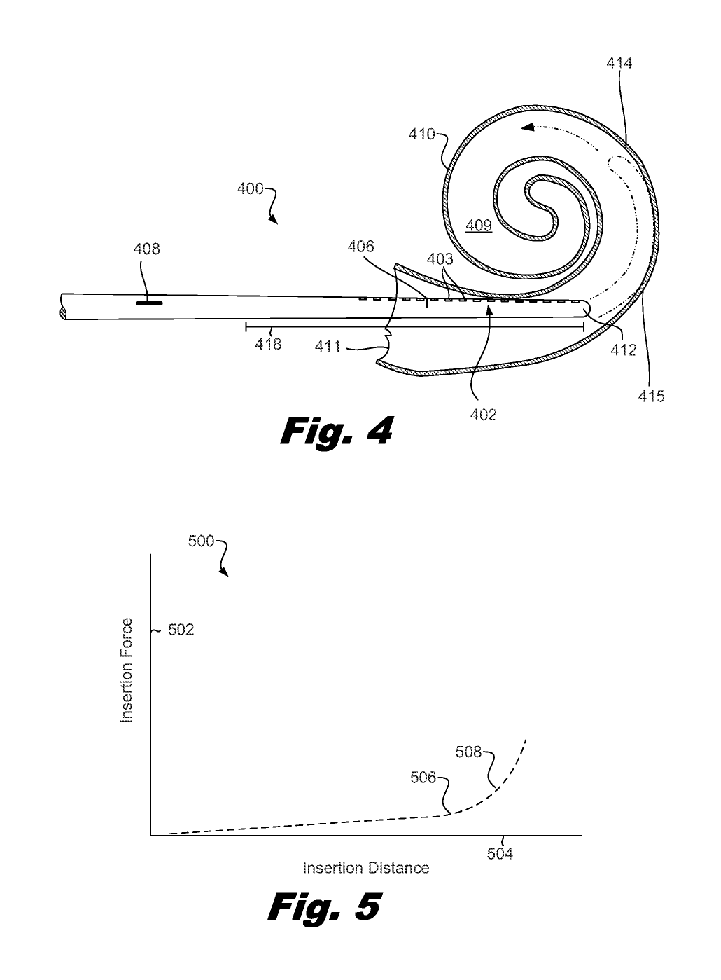

FIG. 4 is a diagram of an example of an implantable device (400) during an insertion procedure. As shown in FIG. 4, the implantable device (400) flexibly follows the lateral wall of the cochlea (410) during insertion so as to conform to the spiral shape of the cochlea and position electrodes (403) proximate to corresponding nerve cells to be stimulated. The point toward the bottom of FIG. 4 at which the cochlea begins to curve is called the basal turn (415).

Determining the proper insertion depth for a cochlear implant is complicated by the fact that different patients will have differently sized cochlea. Based on age and size generally, larger people may have a significantly longer lateral wall and larger cochlea. The variation in total lateral wall length between different patients may indicate different insertion depths to properly place the implant.

However, it has been discovered that the variability of lateral wall length after the variation in basal turn length in patients generally is less than the overall variability of lateral wall length. Accordingly, the present specification announces a method and cochlear implant with which the condition of full or proper insertion can be determined by having a flexural inflection point or discontinuity in the body of the implant corresponding to the location of the basal turn when the implant is fully or properly inserted. This will significantly reduce the angular insertion depth variation between multiple cochlea sizes.

For example, the inflection point (406), described in more detail below, may be placed 15 mm from the distal tip (412) of the implant (400). This may correspond to the location of the twelfth of 16 electrodes (403) from the distal tip (412).

Assuming a length of the lateral wall to be 120 degrees, we assume the length of the array of electrodes will be an additional 15 mm. Based on empirical research, for an average sized cochlea, this predicts an insertion depth of about 420 degrees. Inserting a fixed length after 120 degrees of insertion removes the dependency on the basal length and also reduces by approximately 50% variability due to cochlea size. This technique also compensates for and reduces dependency of insertion depth on the surgical approach, whether via the round window or cochleostomy. Around the insertion depth for an average cochlea of 520 degrees, this method predicts insertion depths of 405 degrees for a large cochlea and 470 degrees for a small cochlea, for a variation in insertion depth of 65 degrees. Thus, this approach could significantly reduce the variability of insertion depth for a lateral wall electrode.

The flexural inflection point (406) is a point along the length of the flexible electrode array at which the array is more flexible, or less stiff, than in portions immediately to either side of the inflection point along the array. A number of techniques, as disclosed herein, may be used for creating such an inflection point, including a notch or slit in, or decreased width of, the flexible electrode array body at the inflection point. Alternatively, the inflection point may be formed by replacing a portion of the material used to form the flexible electrode array body with a different, more flexible material. Thus, the flexible electrode array body has an increased ability to bend at each flexural inflection point than at other points along the length of the flexible electrode array. The notch or other flexural feature can be formed between electrodes or on the non-modiolar side of the array body opposite the electrodes.

A flexible electrode array having a flexural inflection point, as described, provides a number of advantages. For example, the flexible electrode array may have a single flexural inflection point which is located at a position along the flexible electrode array that will, upon insertion, reside at the basal turn of the cochlea. The inflection point decouples the straightening tendency or bias of the distal portion of the array from the portion residing in the basal turn. This allows the flexible electrode array to more readily conform to the shape of the cochlea with less tendency to move over time relative to its initial placement in the cochlea.

Absent the proposed inflection point, a straightening force may cause a flexible electrode array to press against the walls of the cochlea, and increase movement and migration of the flexible electrode array. With the proposed flexural inflection point, the straightening forces resulting from the distal portion of the array pressing against the wall of the cochlea are balanced or absorbed at the inflection point by the array pressing against the wall of the basal turn rather than allowing those forces to act further along the array toward its proximal end at the cochleostomy or insertion window. This reduction in straightening force may also reduce the forces against the cochlea during insertion through the cochleostomy, as well as during use of the implantable device.

Another advantage of this flexural inflection point is that it resists being pushed past the bottom of the basal turn. The same decoupling action of the flexural inflection point that prevents axial forces from being transmitted from the distal to the proximal end of the array also prevent insertion forces from being transmitted into the distal portion beyond the flexural inflection point once the flexural inflection point has reached the beginning of the basal turn. This helps to prevent over-insertion of the array into the cochlea that could cause damage to the cochlea, the electrode array or both. Moreover, as will be further described below, the resistance encountered when, during insertion, the flexural inflection point reaches the basal turn may signal full insertion of the implant.

In other examples, a number of flexural inflection points may be formed along the length of the electrode array to further reduce insertion force, such as friction and internal spring forces, while the flexible electrode array is being inserted in a cochlea. Flexural inflection points along the length of the electrode array will also absorb forces on the array as a patient moves following implantation. This can minimize migration of the implanted electrode array and damage to the cochlea and the array itself. For example, these flexural inflection points may be disposed at 2 to 3 mm apart along the length of the electrode array.

FIG. 4 is a diagram of an example of an implantable device (400) during an insertion procedure. Here, the implantable device (400) is a cochlear implant that has a flexible electrode array (402). A number of electrodes (403) are incorporated into and spaced along the length of the flexible electrode array (402). A flexural inflection point (406) is also provided along the length of the flexible electrode array (402). When inserted into the scala tympani (409), the flexural inflection point (406) is positioned to create a bend of the flexible electrode array (402) to reduce the axial forces transmitted along the flexible electrode array (402) as described above.

During the insertion procedure, a hole is cut into a part of the cochlea (410) to form a cochleostomy through which the flexible electrode array (402) is inserted. Alternatively, a hole may be cut into the round window through which the array may be inserted. Friction may occur at the interface of the hole and the surface of the flexible electrode array (402). Also, the length (418) and surface area of the flexible electrode array (402) in contact with the internal wall (414) of the cochlea (410) generates friction. This friction increases as more of the length (418) makes contact with the internal wall (414) and progressively increases during the insertion procedure.

As the inflection point (406) reaches the beginning of the basal turn, there will be an increase in insertion resistance as the electrode array bends at that inflection point thereby decoupling to some degree the insertion force from the distal end of the electrode array that is beyond the basal turn and beyond the inflection point that has now arrived at the basal turn. The surgeon or other practitioner inserting the implant can notice this relatively sharp increase in insertion resistance to know that the inflection point (406) has arrived at the basal turn and that the implant is accordingly fully inserted.

Additionally, to accommodate the varying sizes and shapes of patient cochlea, the flexible electrode array (402) may include one or more depth markers (408) to indicate a range of minimum insertion depths that will be suitable for larger to smaller cochleae. According to one illustrative example, marker (408) may be located proximal of the electrodes, and may comprise the same material as the electrodes but not be electrically connected to any wires. Such a marker might be used in conjunction with the flection point (406) such that the increase in insertion resistance, described above, when the inflection point (406) arrives at the basal turn occurs somewhere along the depth marker (408) indicating a position between a minimum and maximum insertion depth.

The proximal end of the depth marker (408) may be used to indicate a maximum insertion depth of the implantable device (400). Thus, the professional inserting the electrode array will want to position the electrode lead at an insertion depth between the maximum and minimum indicated by the marker (408) and at a position where the insertion resistance increases sharply indicating that an inflection point (406) is located at the beginning of the basal turn. In this way, the depth marker (408) may help, with the flexural inflection point (406) avoid over-insertion of the flexible electrode array (402). As noted, a flexible electrode array (402) that is over-inserted may cause excessive trauma by tearing the basilar membrane or trans-locating electrodes to the wrong scala.

FIG. 5 is a graph (500) depicting the insertion forces of an implantable device (FIG. 4, 400) according to one example of principles described herein. In the graph (500), the y-axis (502) represents the force exerted on the cochlea (FIG. 4, 410) during insertion of the flexible electrode array (FIG. 4, 402 through the round window (FIG. 4, 411) into the scala tympani (FIG. 4, 409). The x-axis (504) represents the vertical travel, which translates into the depth of insertion of a flexible electrode array (FIG. 4, 402) inside the scala tympani (FIG. 4, 409). Line (506) schematically represents how the force needed to push the flexible electrode array (FIG. 4, 402) deeper into the cochlea (FIG. 4, 410) increases as the insertion distance increases. At point (508), the schematic depicts a sharp increase in the amount of force needed to push the flexible electrode array (FIG. 4, 402) deeper. This point (508) represents inserting a flexural inflection point (FIG. 4, 406) past the basal turn (FIG. 4, 415) of the cochlea (FIG. 4, 410). This increase in force indicates a flexural inflection point (FIG. 4, 406) has passed the basal turn (FIG. 4, 415) of the cochlea (FIG. 4, 410), the flexible electrode array (FIG. 4, 402) is correctly positioned, and force should no longer be applied.

As generally depicted, a sharp increase in the relationship between the insertion force and the insertion distance occurs when inserting the flexible electrode array (FIG. 4, 402) past the basal turn (FIG. 4, 415). This increase represents a portion of the flexible electrode array (FIG. 4, 402) coming in contact with the basal turn (FIG. 4, 415) of the cochlea (FIG. 4, 410). The increase indicates that no further insertion force should be applied.

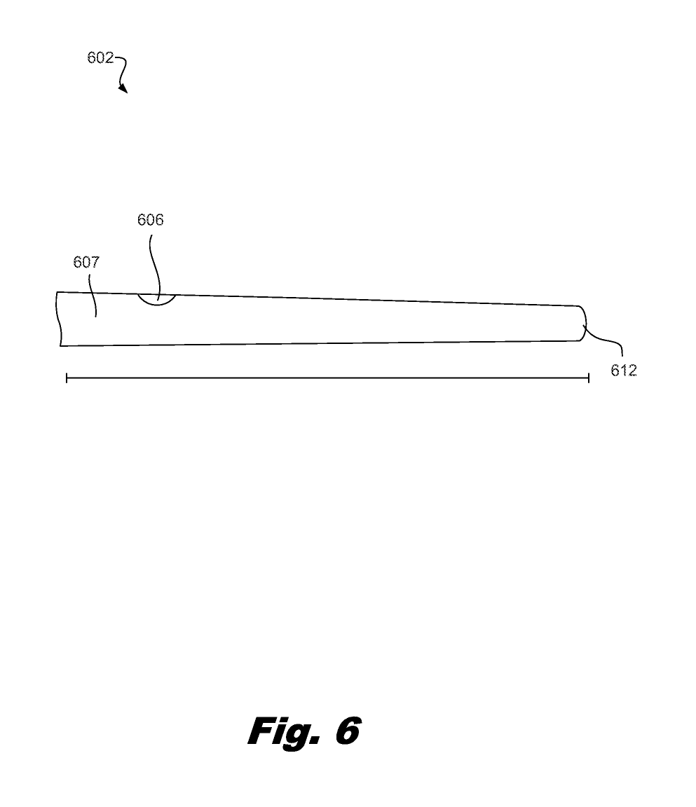

The inflection point (FIG. 4, 406) may be formed using any number of methods, a few non-limiting examples are given below in FIGS. 6-8. These examples are intended to be illustrative in nature. These examples are not intended to be exhaustive or to limit these principles to any precise form disclosed. Many modifications and variations are possible in light of the above teaching.

FIG. 6 is a diagram of a flexible electrode array (602) with a first flexural inflection point (606) according to one example of principles described herein. More specifically, FIG. 6 illustrates a flexible electrode array (602) with a first flexural inflection point (606) at the point where the flexible electrode array (602) should contact the basal turn (FIG. 4, 415). In some examples, the electrode array may have only the single, first flexural inflection point placed in the electrode array so as to be positioned at the basal turn upon implantation of the array.

The first flexural inflection point (606) may be formed by replacing a portion of the material used to form the flexible electrode array (602) body with a different material. For example, a section of the flexible electrode array (602) body may be removed and replaced with a more flexible material to create a flexural inflection point (606) that reduces the stiffness of the flexible electrode array (602). For example, the electrode array body may be made of silicone and the flexural inflection point of a less stiff silicone.

Varying the material composition, for example by using a material with greater flexibility than other areas of the flexible electrode array (602), may allow for controlled bending of the flexible electrode array (602). Placement of the first flexural inflection point (606) may allow the flexible electrode array (602) to bend in a direction following the shape of the cochlea while retaining some stiffness to hold the electrode array in place to stimulate the scala. While FIG. 6 depicts only a first flexural inflection point (606) that includes a more flexible silicone material, as explained above, the device may include any number of other flexural inflection points (606) to improve the bending effect of the flexible electrode array (602).

The first flexural inflection point (606) may decouple the straightening force of the flexible electrode array (602) between the proximal end (607) and the distal end (612). The decoupling may prevent the axial force from being exerted up the flexible electrode array (602) and moving the flexible electrode array (602) out the round window (411, FIG. 4). In other words, the flexural inflection point (606) may prevent dislocation of the flexible electrode array (602), particularly after implantation when the natural movement of the patient may tend to cause the electrode array to migrate. The proximal end (607) may be constructed with additional rigidity to provide an indication that the flexible electrode array (602) has reached a predetermined insertion depth.

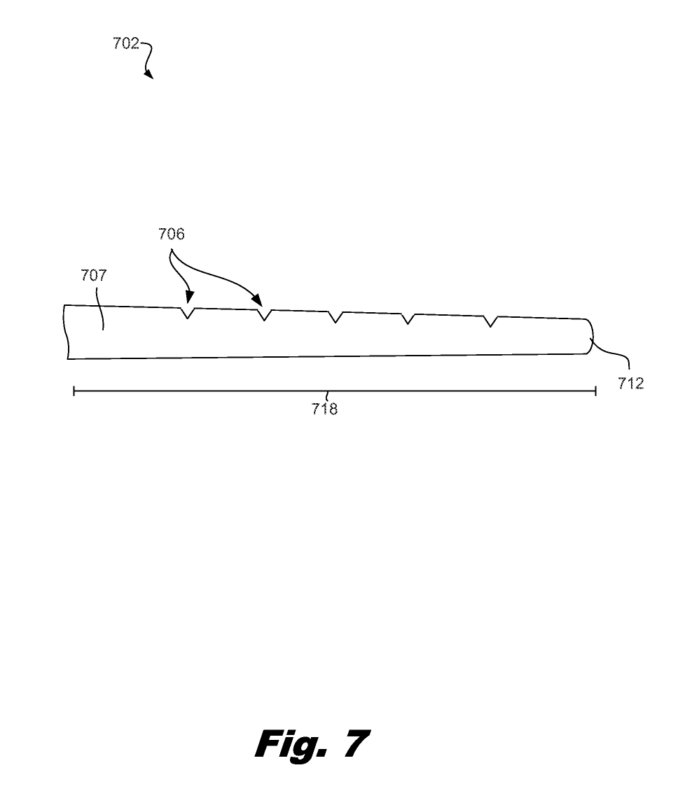

FIG. 7 is a diagram of a flexible electrode array (702) with a number of flexural inflection points (706) according to another example of principles described herein. More specifically, FIG. 7 illustrates a flexible electrode array (702) with a number of flexural inflection points (706) along a length (718) of a distal end (712) of the flexible electrode array (702). The flexural inflection points (706) may be formed by removing a portion of the material used to form the flexible electrode array (702). For example, portions of the flexible electrode array (702) body may be removed to create notches that are flexural inflection points (706) that reduce the stiffness of the flexible electrode array (702) in that section. The removal may allow for the control of the flexural inflection points (706) to provide flexibility in a direction, while maintaining stiffness in other directions. A proximal end (707) may have greater stiffness than the distal end (712). The notch or other flexural feature can be formed between electrodes or on the non-modiolar side of the array body opposite the electrodes.

In such an example, insertion resistance will increase each time an inflection point (706) arrives at the basal turn. Application of further insertion force will push that inflection point (406) around the basal turn. After this, resistance to the insertion force will decrease until the next inflection point arrives at the basal turn. Consequently, the professional inserting the implant may use a depth indicator, such as that described above (408, FIG. 4) to ascertain which increase in insertion resistance is caused by the arrival at the basal turn of the specific inflection point positioned to indicated full insertion, e.g., 15 mm from the distal tip of the implant. This action can be used to help place the electrode array at a correct depth in the cochlea without over-insertion.

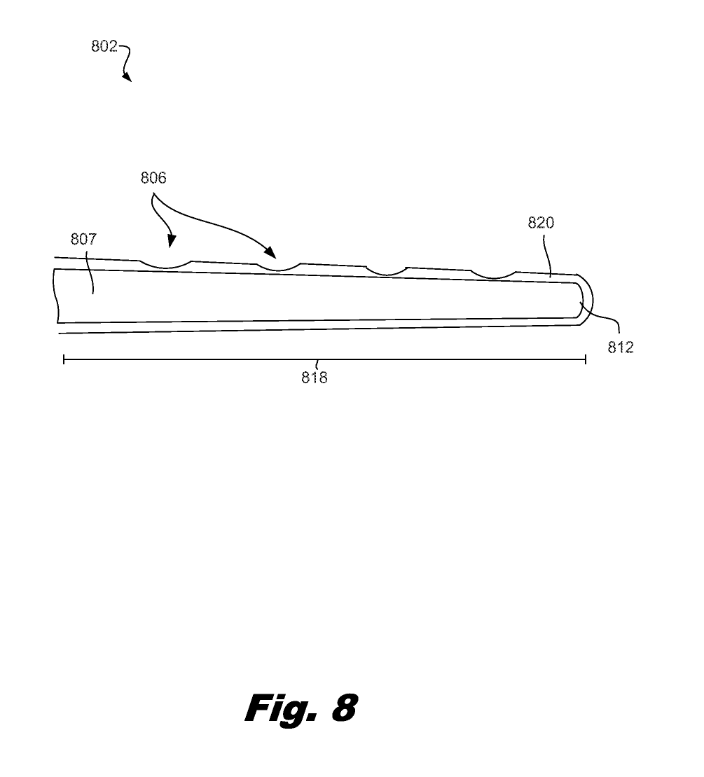

FIG. 8 is a diagram of a flexible electrode array (802) with a number of flexural inflection points (806) according to another example of principles described herein. More specifically, FIG. 8 illustrates a flexible electrode array (802) having a coating (820) over a portion of the flexible electrode array (802). The coating (820) may contain variations in material, cross-sectional shape, and thickness, among other characteristics to create the number of flexural inflection points (806). At least one flexural inflection point (806) may be placed at the point where the flexible electrode array (802) contacts the basal turn (FIG. 4, 415) of the cochlea (FIG. 4, 410). The proximal end (807) of the flexible electrode array (802) may be stiffer than the flexural inflection point (806) by using a coating (820) with a different material, cross sectional shape, and thickness, among other characteristics relative to the coating (820) to form the flexural inflection point (806).

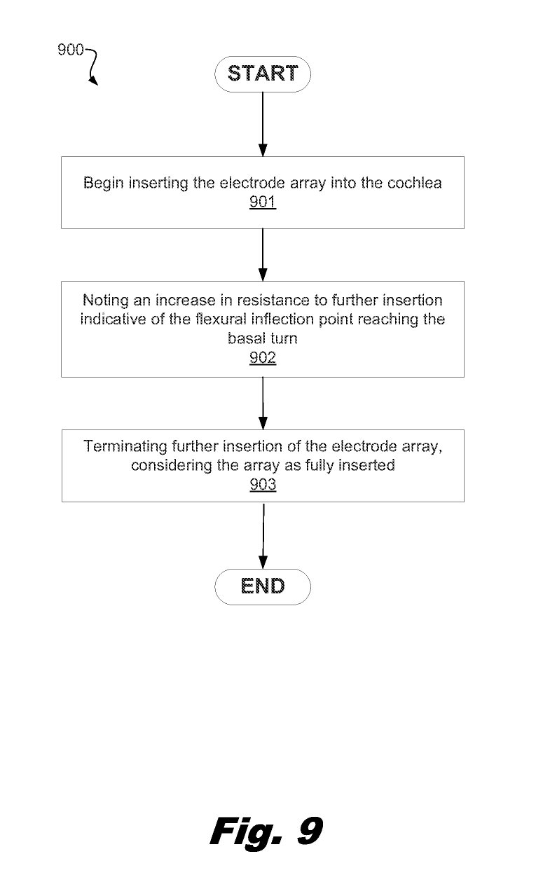

FIG. 9 is a flow diagram of a method for implanting a lead of an implantable device with flexural inflection point according to one example of principles described herein. As shown in FIG. 9, and as elsewhere described herein, the surgeon or other professional begins inserting the electrode array into the cochlea (901).

When the lead bends at the flexural inflection point because the flexural flection point reaches the basal turn, the resistance to further insertion will increase. This increase in resistance is noted by the surgeon as indicating that the flexural inflection point has reached the basal turn (902).

The surgeon then terminates further insertion of the electrode array (903). This is because the flexural inflection point arriving at the basal turn indicates that the array is fully inserted.

The preceding description has been presented only to illustrate and describe examples of, and examples of the principles, described. This description is not intended to be exhaustive or to limit these principles to any precise form disclosed. Many modifications and variations are possible in light of the above teaching.

What is claimed is:

1. A cochlear implant device comprising:
a flexible electrode array for insertion into a cochlea, the flexible electrode array having a proximal end and a distal end;
wherein the flexible electrode array comprises:
a number of electrodes;
a number of electrical wires coupled to the electrodes; and
a flexural inflection point on the flexible electrode array, the flexural inflection point positioned on the flexible electrode array such that, when implanted, the flexural inflection point is situated at a basal turn of the cochlea.

2. The device of claim 1, further comprising a number of additional flexural inflection points between said flexural inflection point and the distal end of the flexible electrode array.

3. The device of claim 1, further comprising a silicone coating surrounding the number of electrical wires, wherein the coating at the flexural inflection point has a different thickness, cross-sectional shape, or combinations thereof, than the coating elsewhere on the flexible electrode array.

4. The device of claim 1, wherein:
the distal end and the proximal end are constructed of a first material; and
at least a portion of the first flexural inflection point is constructed of a second material that is distinct from the first material.

5. The device of claim 4, wherein the second material is more flexible than the first material.

6. The device of claim 1, wherein a profile of the flexible electrode array varies to form the flexural inflection point.

7. The device of claim 1, wherein the flexural inflection point is located about 15 mm from a distal tip of the flexible electrode array.

8. The device of claim 1, wherein the flexible electrode array comprises 16 electrodes, and the flexural inflection point is located at a 12th electrode from a distal tip of the flexible electrode array.

9. The device of claim 1, wherein the flexible electrode array further comprises a depth marker to indicate a minimum insertion depth within the cochlea.

10. The device of claim 9, wherein distal and proximal ends of the depth marker, respectively, indicate a minimum and maximum insertion depth of the array within the cochlea.

11. The device of claim 1, wherein the first flexural inflection point provides an increase in resistance to further insertion when arriving at a beginning of the basal turn.

12. A method of using a cochlear implant device comprising
a flexible electrode array for insertion into a cochlea, the flexible electrode array having a proximal end and a distal end;
wherein the flexible electrode array comprises:
a number of electrodes;
a number of electrical wires coupled to the electrodes; and
a flexural inflection point positioned on the flexible electrode array such that, when implanted, is situated at a basal turn of the cochlea;
the method comprising:
(a) inserting the flexible electrode array into the cochlea while sensing the insertion resistance of the flexible electrode array;
(b) determining when the flexural point reaches the basal turn of the cochlea as indicated by an increase in insertion resistance caused by bending of the flexible electrode array at the flexural inflection point; and
(c) discontinuing further insertion of the flexible electrode array.

13. The method of claim 12, wherein the flexural inflection point is located about 15 mm from a distal tip of the flexible electrode array.

14. The method of claim 12, wherein:
the flexible electrode array comprises 16 electrodes, and
the flexural inflection point is located at a 12th electrode from a distal tip of the flexible electrode array.

15. The method of claim 12, wherein the flexible electrode array comprises a plurality of flexural inflection points, the method further comprising noting an increase in insertion resistance caused by each of said flexural inflection points reaching the basal turn of the cochlea.

16. The method of claim 15, further comprising using a marker indicating maximum and minimum insertion depth for the flexible electrode array to determine which increase in insertion resistance is caused by bending of the flexible electrode array at said flexural inflection point which indicates full insertion of the array; and, then, discontinuing further insertion of the flexible electrode array.

17. The method of claim 12, further comprising forming the flexural inflection point with a silicone coating surrounding the number of electrical wires, wherein the flexural inflection point has a different thickness, cross-sectional shape, or combinations thereof, than the coating elsewhere on the flexible electrode array.

18. The method of claim 12, further comprising forming the flexural inflection point with from a first material different from a second material used to form surrounding portions of the flexible electrode array.

19. The method of claim 18, wherein the first material is more flexible than the second material.

20. The method of claim 12, further comprising forming the flexural inflection point with a profile of the flexible electrode array varies to form the flexural inflection point.
